(19) United States
(12) Patent Application Publication
(10) Pub. No.: US 2007/0154452 A1
(43) Pub. Date: Jul. 5, 2007

(54) Therapeutic agent for pancreas cancer

(75) Inventor: Akikuni Yagita, Tokyo (JP)

Correspondence Address: JHK LAW, P.O. BOX 1078, LA CANADA, CA, 91012-1078, US

(73) Assignee: ORIENT CANCER THERAPY, Tokyo (JP)

(21) Appl. No.: 10/597,206

(22) PCT Filed: Jan. 26, 2005
(86) PCT No.: PCT/JP05/00964

§ 371 Date: Jul. 20, 2006

(30) Foreign Application Priority Data
Jan. 27, 2004 (JP) 2004-018203

(51) Int. Cl. A61K 38/20; A61K 31/7072; A61K 31/716; G01N 33/574

(52) U.S. Cl. 424/85.2; 514/49; 514/54; 435/7.23

(57) ABSTRACT

The present invention is aiming to provide further advantageous effects on treating pancreas cancer, thus providing the means therefor is the subject. The present invention was obtained by finding that there were differences in prognosis in accordance with intrinsic IL-12-producing ability when Novel Immunotherapy for Cancer (NITC) was conducted in targeting pancreatic cancer cases, and further finding that extremely high effects could be obtained on pancreatic cancer therapy by selecting a therapy based on examination results of the level of intrinsic IL-12-producing ability. Therefore, the present invention is to provide an examination method to predict prognostic effects in immunotherapy for pancreas cancer, comprising determination of intrinsic IL-12-producing ability, and a therapeutic agent for pancreas cancer based on that.

### TECHNICAL FIELD

The present invention will provide a new area for pancreatic cancer therapy. Thus the present invention is to provide a novel means for a method for preventing and treating pancreas cancer.

This application claims the priority of Japanese Patent Application No. 2004-018203, which is incorporated herein by reference.

### BACKGROUND ART

Previously, as an innovative approach in cancer therapy, the present inventor Yagita, M. D., focused on the usefulness of a substance which induces interleukin 12 (IL-12) in vivo, discovered that a processed mycelium of mushroom had that function, and established a cancer therapy so called Novel Immunotherapy for Cancer (NITC). There is a fact that conventional IL-12 has anticancer effects but when directly administered in vivo, it provides side effects, making the therapy unacceptable for patients, thus IL-12 itself could not be used as an anticancer agent. However, a formulation comprising the processed mycelium of mushroom reported by Yagita achieved remarkable therapeutic and life-prolonging effects in cancer therapy. In summary, Yagita achieved a goal of cancer therapy by administering an effective amount of a processed mycelium of mushroom enough for inducing IL-12 in vivo (patent document 1).

IL-12 has effects of activating and enhancing killer T cells by the route of TNFα→IFNγ→IL-12→CTL activities. Therefore, anticancer effects are expected to obtain by increasing IL-12 production through activation and enhance of killer T cells.

While pancreas cancer develops both in head and tail of pancreas, they often develop in head thereof. It is really difficult to diagnosing pancreas cancer, so that the diagnosis is conducted, for example, by measuring tumor markers such as CA19-9 and CEA in serum, by ultrasonography, CT (computed tomography) examination or duodenum endoscopic observation, further by endoscopic retrograde pancreatography wherein pancreatic duct is filled with a contrast medium to take X-ray images. Alternatively, the diagnosis is conducted by collecting pancreatic juice to find cancer cells, tumor markers or abnormal genes therein. To treat pancreas cancer, typically pancreas including focus is resected by surgery. When jaundice is serious, the treatment of percutaneous transhepatic cholangio drainage (drainage) is taken to alleviate jaundice first before resection. In case the resection of focus is impossible, a flow pass for bile may sometimes be placed. Then, radiation and the administration of anticancer drugs are carried out. As a therapeutic agent for pancreas cancer, there is gemcitabine hydrochloride (Gemzar™) which is an anticancer agent developed by Eli Lilly and Company and approved to apply for insurance for pancreas cancer in April in 2001. Among malignant tumors, pancreas cancer is believed to bring about the worst prognosis.

(patent document 1) Japanese Patent Application Laid-open No. HEI 10-139670

### DISCLOSURE OF THE INVENTION

(Problems to be Solved by the Invention)

The present invention is aiming to provide advantageous effects on treating pancreas cancer, thus providing the means therefor is the subject.

(Means to Solve the Problem)

The present inventors found that there were differences in prognosis in accordance with intrinsic IL-12-producing ability when Novel Immunotherapy for Cancer (NITC) was conducted in targeting pancreatic cancer cases, and further found that extremely high effects could be obtained on pancreatic cancer therapy by selecting a therapy based on examination results of the level of intrinsic IL-12-producing ability, thereby completed the present invention.

Therefore, the present invention consists of:
1. An examination method to predict prognostic effects in immunotherapy for pancreas cancer, comprising determination of intrinsic IL-12-producing ability.
2. The examination method according to the preceding aspect 1, wherein the prognostic effects are predicted by dividing the IL-12-producing ability into a plurality of, i.e. at least three groups of 50 pg/ml or more, from 7.8 to less than 50 pg/ml, and less than 7.8 pg/ml.
3. The examination method according to the preceding aspect 1 or 2, wherein the immunotherapy is an IL-12 production-inducing agent.
4. The examination method according to any one of the preceding aspects 1 to 3, wherein the IL-12 production-inducing agent is a substance having a β-1,3/1,6 glucan structure.
5. An IL-12 production-inducing agent, wherein the IL-12 production-inducing agent is administered to a pancreatic cancer patient having IL-12-producing ability of less than 7.8 pg/ml in the examination according to any one of the preceding aspects 1 to 4.
6. A cancer therapeutic agent comprising Gemcitabine Hydrochloride as principal component, wherein the agent is used at least in combination with an IL-12 production-inducing agent.
7. The cancer therapeutic agent according to the preceding aspect 6, wherein the cancer is pancreas cancer.
8. The cancer therapeutic agent according to the preceding aspect 6 or 7, wherein the IL-12 production-inducing agent is administered to a pancreatic cancer patient having IL-12-producing ability of less than 7.8 pg/ml.
   (Effects of Invention)

In the present invention, it is suggested that the prognosis of a patient with pancreatic cancer in NITC would be defined by IL-12-producing ability, so that enhancing that IL-12-producing ability is important in immunotherapy.

### BRIEF DESCRIPTION OF THE DRAWINGS

FIG. 1 shows IL-12 levels and survival rates of patients with pancreas cancer.

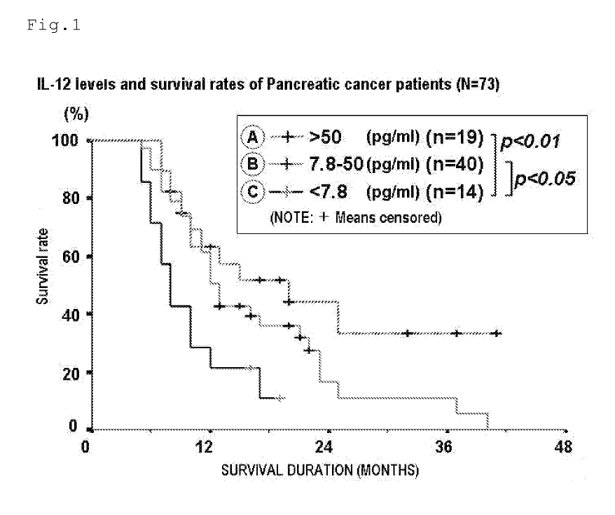

FIG. 2 shows both survival rates in pancreas cancer between a group administered Gemzar alone and a group administered Gemzar and NITC in combination.

FIG. 3 shows changes in Th1 cytokines (IFNγ, IL-12 and Th1/Th2) in pancreas cancer before and/or after the administration of Gemzar.

FIG. 4 shows changes in the ratios of NK cells, perforin-producing NK cells, NKT cells and perforin-producing NKT cells in pancreas cancer before and/or after the administration of Gemzar.

### DESCRIPTION OF THE PREFERRED EMBODIMENTS

The present invention will be described in detail below, and technical and scientific terms used herein have meanings usually understood by those of ordinary skilled in the art unless otherwise specified.

Novel Immunotherapy for Cancer (NITC) provided by the present inventor Yagita, M. D., is a therapeutic means made up by combining four different mechanisms of action.

The first mechanism of action is a method for reducing cancer by administering an angiogenesis inhibitor (better shark) to block blood flow entering cancer. The effects of that can be determined by measuring vascular endothelial growth factor (VEGF). The effects of inhibiting angiogenesis can be evaluated as minus (negative) values of VEGF (−VEGF). Instead of these VEGF values, ability inhibiting angiogenesis can also be evaluated using other vascular endothelial growth factors such as FGF and HGF. Alternatively, its evaluation can be conducted by using the positive values of factors inhibiting angiogenesis (for example, endostatin values) instead of VEGF's.

The second mechanism of action is a method for inducing Th1 cytokines (TNFα, IFNγ and IL-12) by administering a compound carrying β-1,3 glucan structure, thereby activating CTLs. While CTL activity can be determined by perforin-producing ability of CD8 (+), CD8 (+) perforin values are based on two types, cytotoxic T cell (CTL) or suppressor T cell (STC), and the former damages cancer cells, while the activation of the latter leads to the proliferation of cancer in the end. Thus, its absolute value cannot be used in evaluation. However, if IFNγ value is 10 IU/ml or more or IL-12 value is 7.8 pg/ml or more, it is CTL, while both IFNγ and IL-12 values are low, it can be determined STC. Therefore, CTL activity can be evaluated by IFNγ-producing ability (IFNγ value) or IL-12-producing ability (IL-12 value).

Effector cells activated by the administration of a compound carrying an α-1,3 glucan structure are NK and NKT cells, which are the third and fourth mechanisms of action. These NK and NKT cells share NKR-P1 (CD161 (+), receptor of NK cell), and the former NK cell has surface markers of CD3 (−) and CD161 (+), thereby its cell number can be measured, and further its activation can be determined by perforin-producing abilities of CD3 (−) and CD161 (+). On the other hand, the latter NKT cell has CD3 (+) and CD161 (+), thereby its cell number can be measured, and further the activation of NKT cell can also be measured by their perforin-producing abilities (referred to as NKTP(+)).

Therefore, even in Novel Immunotherapy for Cancer (NITC) or in a general immunotherapy, effector cells or the effects inhibiting angiogenesis can be evaluated respectively by the following measuring items. In particular, CTL activity can be evaluated by IFNγ or IL-12 production-inducing ability. The activation of NK cells can be evaluated by either CD3 (−) and CD161 (+), or perforin values of CD3 (−) and CD161 (+). Further, the activation of NKT cells can be evaluated by either CD3 (+) and CD161 (+), or perforin values of CD3 (+) and CD161 (+) (NKTP value).

As an IL-12 production-inducing agent for use in the present invention, for example, a compositional formulations of mushroom mycelium having a β-1,3 glucan structure (for example, ILX™from Touzai Iyaku Kenkyusho K. K.; ILY™from Seishin Enterprise Co., Ltd.), or a variety of yeasts having a β-1,3 glucan structure (marine and bread yeasts, NBG™) can be used. In particular, marine yeasts are preferred. An IL-12 production-inducing agent for use in the present invention will be applied in accordance with a prescription allowing the induction or enhancement of its production-inducing activity, and further the maintenance of that activation as well. Thus, the agent will be used by selecting dosage and administration period which can induce or enhance its activation, and further maintain that activation. In particular, the dosage of a compound having a β-1,3 glucan structure, which is a CTL activator (IL-12 production-inducing agent or INFγ production-inducing agent), may be from about 1 to 10 g/day, and preferably from about 3 to 6 g/day. Further, the administration period may usually be between for 10 days and 24 months, and the administration frequency may be on alternate days, or from one to three times a day, and daily administration is preferred. The IL-12 production-inducing agent of interest will preferably be administered orally.

Therapeutic Effects of NITC Therapy in Pancreas Cancer

When Novel Immunotherapy for Cancer (NITC) is targeted to pancreatic cancer cases, there are significant differences seen in prognosis caused by intrinsic IL-12-producing ability. A group obtaining the best prognosis was Group A (15 cases) (50 pg/ml ≦IL-12 producing ability). Then, the others were as follows: Group B (40 cases) (7.8 ≦IL-12 producing ability <−50 pg/ml) and Group C (14 cases) (IL-12 producing ability <7.8 pg/ml). The survival rates showed a significant difference (p<0.01) between Groups A and C, and a significant difference of survival rate was also confirmed between Groups B and C (p<0.05). These results suggested that prognosis of a patient with pancreatic cancer subjected to NITC would be defined by IL-12-producing ability, so that enhancing that IL-12-producing ability was important in immunotherapy. In summary, it means that selecting an IL-12 production-inducing agent is important.

In the present invention, as an immunotherapeutic agent for cancer, besides an IL-12 production-inducing agent, NK or NKT activator can be used. As an NK or NKT activator, a compositional formulation of a compound having an α-1,3 glucan structure such as nigerooligosaccharide and fucoidan is useful. A variety of compounds having an α-1,3 glucan structure are known, thus combining this known structure with the measurement of CD3 (−) and CD161 (+) perforin producing abilities of CD3 (−) and CD161 (+), CD3 (+) and CD161 (+), and perforin producing abilities of CD3 (+) and CD161 (+) will allow those skilled in the art to easily determine which NK activator to be used. Meanwhile, CD3 (+) and CD161 (+) mean they affect the receptor NKR-P1 of NKT cell.

Effects of Combination of Gemcitabine Hydrochloride with NITC

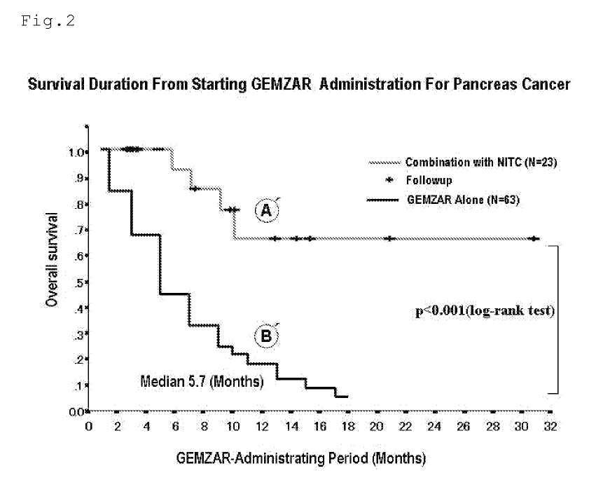

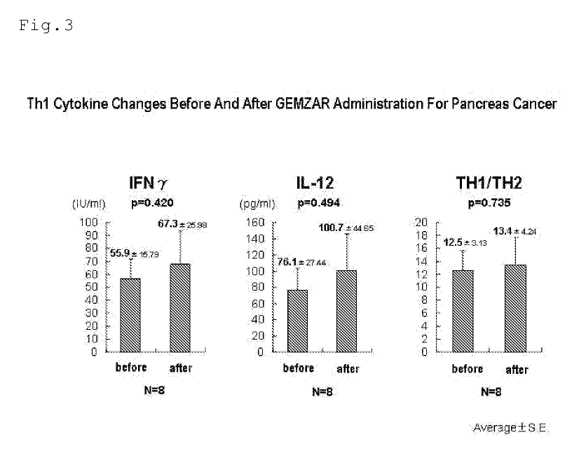

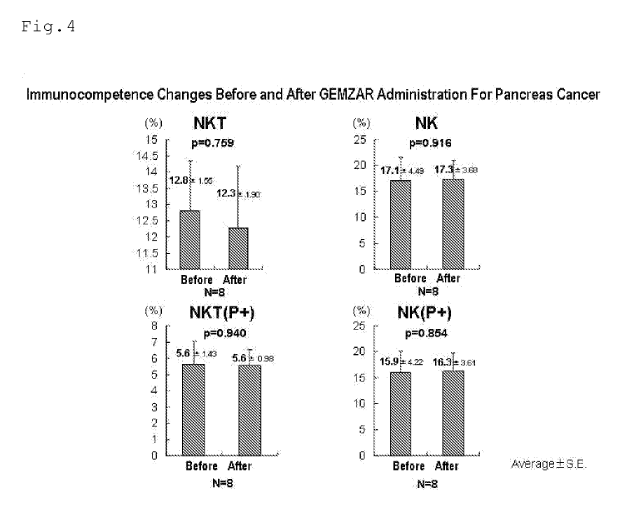

Gemcitabine hydrochloride (Gemzar™) is an anticancer agent developed by Eli Lilly and Company. This Gemzar was approved to apply for insurance for pancreas cancer in April in 2001. FIG. 2 shows survival rates of a group administered with Gemzar alone (63 cases) (Group B′)and a group administered with Gemzar and NITC in combination (23 cases) (Group A′). Comparing the prognoses between these two groups, Group A′ was clearly more effective than Group B′, and a significance probability in Log-rank test at the three time points in 6, 9 and 12 months was p<0.001. Further, the examination of effects inhibiting immunocompetence before and after the administration of Gemzar (1000 mg/mm² was administered continuously for three weeks followed by a week break) revealed that effects inhibiting cytokines provided by Gemzar administration were not observed on any of Th1 cytokines of IFNγ (8 cases), IL-12 (8 cases) and Th1/Th2 (8 cases) (FIG. 3). Inhibitory effects were also not observed in the ratios of NK cells (8 cases), perforin-producing NK cells (8 cases), NKT cells (8 cases) and perforin-producing NKT cells (8 cases) to total lymphocytes (FIG. 4). While other anticancer agents administered at a normal dosage showed significant effects inhibiting Th1 cytokines, thus this is believed to be a reason why the combination of NITC and Gemzar can be effective on pancreas and bile duct cancers. Meanwhile, Gemzar has also been approved for non-small cell lung cancer, and the same may be suggested for lung cancer and others.

Usually, when the combination therapy of anticancer (chemotherapy) agent, radiation or steroid is further added to the present combination therapy, the route of TNFα→IFNγ→IL-12→killer T cell is significantly interfered of two routes of immune systems. Therefore, they will preferably not be used except for Gemzar. However, when an anticancer agent is administered, application of low concentration chemotherapy, which is an administration method never interfering with immune systems described above, is useful, i.e., the administration of 5 FU, UFT, Mifurol, furtulon, or CDDP at a low concentration (from 5 μg to 10 μg) or an anticancer agent such as Taxotere, Taxol, adriamycin, mitomycin or CPT-11 at a low concentration is useful. Likewise, a low dose irradiation should be applied in radiation therapy and a low concentrate administration be selected in steroid therapy.

Measuring Method of Cells and Each Cytokine will be shown below.

(Measurement of NKT Cells) (Measurement of NK Cells) (Measurement of CD8)

The measurement of NKT cells having NKR-P1 can be conducted by measuring cell surface antigens (CD3 and CD161 ) which are specifically presented on the NKT cell surface. In particular, lymphocytes in peripheral blood are tested for cells of CD3 positive and CD161 positive [CD3 (+) and CD161 (+)]. Thus, CD3 and CD161 which are cell surface antigens on NKT cell are measured by Two Color assay which uses monoclonal antibodies and flow cytometry. Here, activated NKT cells mean that the ratio of NKT [CD3 (+) and CD161 (+)] cells in lymphocytes is 10% or more, and more preferably 16% or more. The ability of activating NKT cell means the function which can increase NKT cell ratio by 10% or more, and more preferably 16% or more, or which can further increase NKT cell ratio more than that before administering a certain substance. Likewise, [CD3 (−) and CD161 (+) ] means to assay for CD3 negative and CD161 positive cells. This method is useful in measurement of NK cells. In addition, CD8 (+) means to assay for CD8 positive cells. This method is useful in measurement of CTL activity.

In the Examples, bloods from cancer patients were used, and cells in blood were separated into positive and negative for cell surface antigens, CD3, CD161 and CD8, and each cell ratio was measured by Two Color assay using flow cytometry following a conventional manner. At this time, monoclonal antibodies used against CD3 , CD161 and CD8 were supplied by Coulter or Becton & Dickinson respectively.

(Measurement of Perforin-Producing cell)

For lymphocytes in peripheral blood, two of CD3 , CD161 and CD8 of cell surface antigens and perforin are measured by Three Color assay using flow cytometry following a conventional manner. Specifically, into a collected blood, a fixative solution is added to fix cells, then after adding a membrane permeabilizer, an anti-perforin antibody (supplied by Pharmingen) is added for reaction, further secondary antibody labeled with PRE-Cy5 (from DAKO) is added for reaction, then anti-CD3-PE (Coulter 6604627) antibody and anti-CD161-FITC (B-D) antibody are added for reaction, thereafter subjected to measurement by flow cytometry. They were abbreviated as P or PER in figures and tables.

(Preparation of Samples for Measuring Cytokines)

Firstly, a monocyte fraction is separated from blood for preparation. After heparinized peripheral blood is diluted twofold with Phosphate Buffer Saline (PBS) and mixed, the mixture is overlaid on Ficoll-Conray solution (specific gravity 1.077), centrifuged at 400G for 20 minutes to collect a monocyte fraction. After washing it, RPMI-1640 medium added with 10% fetal bovine serum (FBS) is added and prepared such that the cell number becomes 1×10⁶. Into 200 μl of the obtained cell suspension, Phytohemagglutinin (supplied by DIFCO) is added into a concentration of 20 μg/ml, then cultured it in a 96-well microplate in the presence of 5% CO₂ at 37° C. for 24 hours to obtain a sample for measuring two kinds of cytokines in the cultured cell solution.

(Measurement of IL-12)

To measure the amount of IL-12, a measuring kit based on enzyme immunoassay (ELISA) available from, for example, R&D SYSTEMS and MBL is used, though well known clinical and biochemical examinations can be used. Herein, a measuring kit supplied by R&D SYSTEMS was used. In practice, into each well of a 96-well microplate, 50 μl of measuring diluent of Assay Diluent RD1F, and 200 μl of standard or sample prepared from the preparation method for measuring cytokines described above were dispensed, and then allowed it to stand and react at room temperature for 2 hours. Thereafter, 200 μl of anti-IL-12 antibody labeled with horse radish peroxidase (HRP) was dispensed therein and allowed to stand for 2 hours at room temperature. The reaction solution was removed from each well and washed three times, then 200 μl of substrate solution for developing color was dispensed, allowed to stand for 20 minutes at room temperature, and then 50 μl of a solution for terminating enzyme reaction was dispensed respectively. The absorbance of each well at 450 nm was measured with Emax (supplied by Wako Pure Chemical Industries, Ltd.), using that of 550 nm as control. The amount of IL-12 is expressed as pg/ml. Herein, IL-12 production inducer ability means the function which can increase the amount of IL-12 produced from the monocyte fraction of peripheral blood by stimulation to 7.8 pg/ml or more, or which can increase the amount of the produced IL-12 more than that before administering a certain substance.

(Measurement of IFNγ)

The measurement of IFNγ was conducted by enzyme immunoassay (EIA method) using IFNγ EASLA kit from BioSource Europe S. In practice, into a each well of a 96-well microplate, 50 μl of standard or the twofold dilution of prepared sample described above was dispensed, then 50 μl of anti-IFNγ antibody labeled with HRP was dispensed, and further allowed to react for 2 hours at room temperature with shaking. The reaction solution was removed from each well and washed three times, then 200 μl of a substrate solution for developing color was dispensed, allowed to react for 15 minutes at room temperature with shaking, and then 50 μl of a solution for terminating enzyme reaction was dispensed. The absorbance of each well at 450 nm and 490 nm was measured with Emax (supplied by Wako Pure Chemical Industries, Ltd.), using that of 630 nm as a control. The amount of IFNγ is expressed as IU/ml.

(Measurement of Ability Inhibiting Angiogenesis)

(Measurement of vascular endothelial growth factor/VEGF, basic fibroblast growth factor/bFGF, and factors inhibiting angiogenesis endostatin/Endostatin) Using commercially available kits, concentrations of the factors in serum were measured by enzyme immunoassay solid phase methods respectively (ELISA; enzyme linked immuno-sorbent assay) (ACCUCYTE Human VEGF, ACCUCYTE Human bFGF, ACCUCYTE Human Endostatin: CYTIMMUNE Sciences Inc.).

Meanwhile, each marker used in the clinical examination was commercial product and the measured values were exhibited according to each recommended manner. Abbreviations expressed were based on general expression ways respectively.

Determination of effects on patients adopted the following five-graded determination: CR (complete remission), PR (partial remission), LNC (long-term no change), SNC (short-term no change) and PD (progressive disease state). Further, the rate of effects in each cancer species means the rates of CR, PR, LNC, SNC and PD to all cases combining those of each cancers species.

### EXAMPLE

The present invention will be described in detail below with using Examples, but the present invention will not be limited by those Examples.

Progressive terminal cancer cases had been treated with Novel Immunotherapy for Cancer (NITC). This NITC is a BRM therapy wherein intrinsic TNFα, IFNγ and IL-12 are induced by administering γ-1,3 glucan, thereby activating CTL (killer T cell), as well as NK and NKT cells are activated by administering α-1,3 glucan, and at the same time angiogenesis is inhibited by administrating better shark. To patients, an immunotherapeutic agent for cancer, an IL-12 production-inducing agent, Shark cartilage (Seishin Enterprise Co., Ltd.), a saccharide having an α-1,3 structure and others were administered according to recommended prescriptions respectively. Meanwhile, as an IL-12 production-inducing agent, ILX (Touzai Iyaku), ILY (Seishin Enterprise Co., Ltd.), Krestin (Sankyo), Immutol (NBG) and the like were administered alone or in combination concerning patients' symptoms.

### Example 1

Case 1. Bile Duct Cancer, 47-Year Old Man, Case Treated with NITC Alone

A case treated with NITC alone and determined CR will be described. This case underwent resection of hepatic portal region on January, 10 in Heisei-1x, following the diagnosis of hepatic portal bile duct cancer, but still pathological diagnosis exhibited the remaining cancer cells at the resected end. NITC was started on February, 1 in Heisei-1x. Tumor markers exhibiting abnormal values at the first medical examination were SLX-1 of 57 IU/ml (normal value≦38) and 1CTP of 13.7 ng/ml (normal value≦4.5). Both values of immunocompetence at that time were lowered, showing IFNγ of 3.1 IU/ml (activation value≧10) and IL-12 value of less than 7.8 pg/ml (activation value ≧7.8 pg/ml). However, two months after starting NITC, IFNγ value was activated to 57.4 IU/ml and IL-12 value to 58.4 pg/ml, while SLX-1 normalized to 32 U/ml and 1CTP decreased to 11.3 ng/ml. Thereafter, both values of IFNγ and IL-12 were sustained active at all times, and 1CTP reached a normal level in a year and three months until May, 24, Heisei-14 (2002), thus the patient was determined “CR”.

### Example 2

Case 2. Bile Duct Cancer, 66-Year Old Man, Case Treated with a Combination of NITC and Gemzar

A case confirming the effects of combination therapy of NITC and Gemzar will be described. Bile duct cancer and multiple hepatic metastasis were observed in this case in February, Heisei-1x. Thereafter, a reservoir for intraarterial injection was placed at hepatic metastatic focuses to administer CDDP and 5 Fu in a different hospital, but effects were not shown. On July, 15 in Heisei-15 (2003), NITC was initiated. The values of Dupan-2, tumor marker (normal value=150 U/ml), were 8900 U/ml on August, 21 and 8300 U/ml on September, 8 in Heisei-1x, showing a poor improvement. Thus, 1000 mg/mm² of Gemzar was administered three times from September, 18. As a result, Dupan-2 exhibited a remarkable improvement of 6110 U/ml on October, 2 in Heisei-1x.

### INDUSTRIAL APPLICABILITY

As described above, according to the examination method of the present invention, prognostic effects can be predicted in an immunotherapy for pancreas cancer, so that based on that prediction, an effective treatment of pancreas cancer can be provided. Further, it was exhibited that the therapeutic agent for pancreas cancer of the present invention could provide high therapeutic effects on patients with pancreas cancer by enhancing their IL-12-producing ability.

1. An examination method to predict prognostic effects in immunotherapy for pancreas cancer, comprising determination of intrinsic IL-12-producing ability.

2. The examination method according to claim 1, wherein the prognostic effects are predicted by dividing the IL-12-producing ability into a plurality of, i.e. at least three groups of 50 pg/ml or more, from 7.8 to less than 50 pg/ml, and less than 7.8 pg/ml.

3. The examination method according to claim 1, wherein the immunotherapy is an IL-12 production-inducing agent.

4. The examination method according to claim 1, wherein the IL-12 production-inducing agent is a substance having a β-1,3/1,6 glucan structure.

5. An IL-12 production-inducing agent, wherein the IL-12 production-inducing agent is administered to a pancreatic cancer patient having IL-12-producing ability of less than 7.8 pg/ml in the examination according to claim 1.

6. A cancer therapeutic agent comprising Gemcitabine Hydrochloride as principal component, wherein the agent is used at least in combination with an IL-12 production-inducing agent.

7. The cancer therapeutic agent according to claim 6, wherein the cancer is pancreas cancer.

8. The cancer therapeutic agent according to claim 6, wherein the IL-12 production-inducing agent is administered to a pancreatic cancer patient having IL-12-producing ability of less than 7.8 pg/ml.

9. The examination method according to claim 2, wherein the immunotherapy is an IL-12 production-inducing agent.

10. The examination method according to claim 2, wherein the IL-12 production-inducing agent is a substance having a β-1,3/1,6 glucan structure.

11. The examination method according to claim 3, wherein the IL-12 production-inducing agent is a substance having a β-1,3/1,6 glucan structure.

12. An IL-12 production-inducing agent, wherein the IL-12 production-inducing agent is administered to a pancreatic cancer patient having IL-12-producing ability of less than 7.8 pg/ml in the examination according to claim 2.

13. An IL-12 production-inducing agent, wherein the IL-12 production-inducing agent is administered to a pancreatic cancer patient having IL-12-producing ability of less than 7.8 pg/ml in the examination according to claim 3.

14. An IL-12 production-inducing agent, wherein the IL-12 production-inducing agent is administered to a pancreatic cancer patient having IL-12-producing ability of less than 7.8 pg/ml in the examination according to claim 4.

15. The cancer therapeutic agent according to claim 7, wherein the IL-12 production-inducing agent is administered to a pancreatic cancer patient having IL-12-producing ability of less than 7.8 pg/ml.
